(19) United States
(12) Patent Application Publication
(10) Pub. No.: US 2024/0226407 A9
(43) Pub. Date: Jul. 11, 2024

(54) PHOTO-ECMO APPARATUS, SYSTEMS, AND METHODS

(71) Applicant: The General Hospital Corporation, Boston, MA (US)

(72) Inventors: Richard Rox Anderson, Boston, MA (US); Warren Zapol, Boston, MA (US); Anna Fischbach, Boston, MA (US); William A Farinelli, Boston, MA (US); Sandeep Korupolu, Boston, MA (US)

(21) Appl. No.: 18/547,969

(22) PCT Filed: Feb. 25, 2022
(86) PCT No.: PCT/US2022/017975

§ 371 Date: Aug. 25, 2023

Related U.S. Application Data
(60) Provisional application No. 63/153,410, filed on Feb. 25, 2021.

(51) Int. Cl. A61M 1/36; A61M 1/16

(52) U.S. Cl. CPC A61M 1/3681; A61M 1/1698; A61M 2202/0233

(57) ABSTRACT

An apparatus for removing CO from blood, the apparatus including: a housing configured to house blood obtained from a body of a subject within an interior of the housing; a plurality of gas-permeable tubules disposed within the interior of the housing; an optical intrusion coupled to the housing and configured to project into the housing, the optical intrusion configured to transmit light into the interior of the housing; and a light source optically coupled to the optical intrusion, the light source being configured to emit light which is coupled via the optical intrusion into the interior of the housing such that the emitted light interacts with the blood from the body of the subject.

### CROSS-REFERENCE TO RELATED APPLICATIONS

The present application is based on and claims priority from U.S. patent application Ser. No. 63/153,410, filed on Feb. 25, 2021, the entire disclosure of which is incorporated herein by reference.

### STATEMENT REGARDING FEDERALLY SPONSORED RESEARCH

N/A

### BACKGROUND

Carbon monoxide (CO) poisoning is harmful and in some cases can be lethal, largely because CO strongly competes with oxygen (O₂) for the gas ligand-binding sites on hemoglobin (Hb). Specifically, these two gas molecules, when inhaled, initially dissolve into blood in the lung then associate (in a forward binding reaction) with any of four binding sites on the heme groups of Hb. The association rate constants for O₂ and CO with Hb are approximately equal and controlled mainly by the rate at which the molecules come together by diffusion. The bound gas molecules also spontaneously disassociate (unbind) from hemoglobin. However, the disassociation rate for HbCO is hundreds of times slower than that for HbO₂. In essence, CO occupies the Hb binding sites that are “intended” for oxygen transport.

### SUMMARY

One embodiment provides an apparatus for removing CO from blood, the apparatus including: a housing configured to house blood obtained from a body of a subject within an interior of the housing; a plurality of gas-permeable tubules disposed within the interior of the housing; an optical intrusion coupled to the housing and configured to project into the housing, the optical intrusion configured to transmit light into the interior of the housing; and a light source optically coupled to the optical intrusion, the light source being configured to emit light which is coupled via the optical intrusion into the interior of the housing such that the emitted light interacts with the blood from the body of the subject.

Another embodiment provides a method for removing CO from blood, the method including: providing a housing configured to house blood obtained from a body of a subject within an interior of the housing, a plurality of gas-permeable tubules disposed within the interior of the housing; transmitting light into the housing using an optical intrusion coupled to the housing and configured to project into the housing; and emitting light into the interior of the housing using a light source optically coupled to the optical intrusion such that the emitted light interacts with the blood from the body of the subject.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A shows a simplified diagram of a veno-arterial (VA)-connected ECMO system which includes an oxygenator connected to a pump, where the pump and oxygenator are connected to the patient's circulatory system using cannulas inserted into a femoral artery and femoral vein.

FIG. 1B shows a cross-sectional diagram of paths of red blood cells (wavy lines) through the gas-permeable tubule array (shown in cross-section as a series of circles) of an oxygenator such as that depicted in FIG. 1A; the view in FIG. 1B is perpendicular to the view shown in FIG. 1A (i.e. in a direction facing into the image) as seen from the side or top.

FIG. 2 shows a schematic diagram of how Optical Intrusion (OI) devices may be incorporated into the housing of a device similar to the oxygenators depicted in FIGS. 1A and 1B to produce a PECMO device; FIG. 2 shows two examples of possible orientations of OIs in such a device, with arrows indicating a direction of light input to the OIs.

FIG. 3 shows a close-up view of a single OI device, such as that depicted in FIG. 2, integrated into a transparent wall of the housing of a PECMO device. FIG. 3 shows a light source (e.g. a high-powered LED or laser) directing light into the transparent wall of the PECMO housing and into the OI, which can be conical, wedge-shaped, or other converging shape. Also shown is the optical field (shaded region adjacent to the wedge-shaped sides of the OI) that is delivered by the OI into the surrounding space that is occupied by material including blood and gas tubules as well as exemplary ray paths (dashed lines).

FIG. 4 shows a cross-sectional view of a PECMO housing which includes multiple OIs coupled to the housing and arrays of light sources (e.g. LEDs or lasers, directly coupled or coupled via waveguides to the housing) that are aligned with the OIs and configured to direct light into each of the OIs. Light is thus directed into the interior of the housing of the PECMO device where it is then emitted from the OIs into the space that is occupied by material including blood and gas tubules.

FIG. 5 provides a diagram showing a cross-section of a housing of a PECMO device with wedge-shaped OIs that are hollow cavities extending into the interior of the housing.

FIG. 6 provides a diagram showing a cross-section of a housing of a PECMO device in which fiber optic OIs extend into the interior of the housing.

FIG. 7 shows an example of a process for removing CO from blood in accordance with some embodiments of the disclosed subject matter.

### DETAILED DESCRIPTION

In accordance with some embodiments of the disclosed subject matter, mechanisms (which can include systems, methods, and media) for removing CO from blood using a photo-ECMO (PECMO) device are provided. These mechanisms take advantage of the fact that absorption of light by HbCO leads to efficient photodissociation of CO from Hb. The quantum energy of a visible light photon is somewhat greater than the bond strength of HbCO which, along with other factors, makes the quantum yield (i.e., the probability of dissociation per absorbed photon) for HbCO photodissociation nearly 1. Absorption of light by HbO₂ also leads to photodissociation but with much lower efficiency, such that the quantum yield for HbO₂ photodissociation is about 0.08. Therefore, sufficient light exposure at wavelengths absorbed by HbCO can preferentially remove CO from the Hb binding sites, making it possible for O₂ to competitively bind to the empty sites.

However, the wavelengths of light that are absorbed by HbCO do not penetrate deeply into the human body and thus are unlikely to reach the lungs in larger animals. In small animals we have determined that light exposure on the external surface of the animal can reach the animal's lungs and rapidly clear CO from the body simply by exhalation. For this procedure to work in large animals including humans, a mechanism is needed to allow light to gain access to blood to promote photodissociation of CO from Hb.

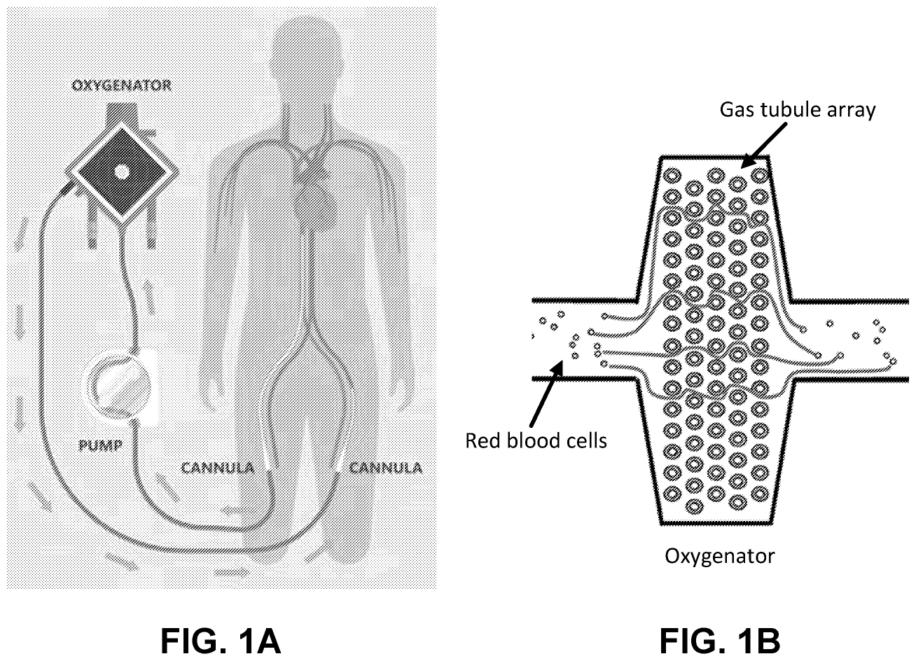

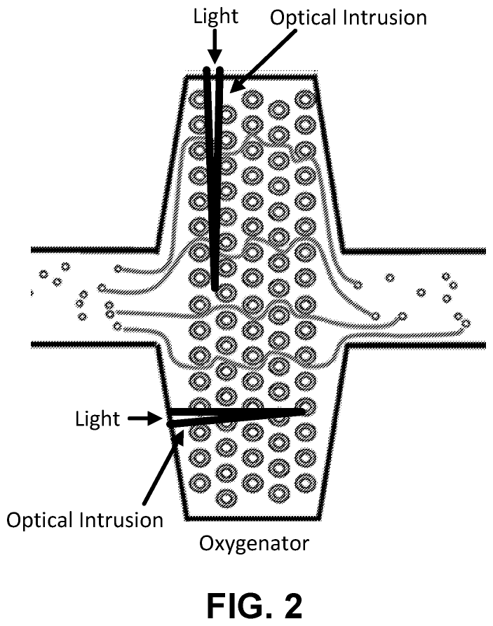

To this end, we have studied the potential for modifying extracorporeal membrane oxygenator (ECMO) devices (see FIG. 1A) to incorporate the use of light for removal of CO. An ECMO device uses an external pump to shunt blood from a patient's body through an oxygenator which promotes exchange of gases from Hb in red blood cells, releasing CO₂ and binding O₂ (FIG. 1A). This exchange is promoted by pumping the blood through a chamber in the oxygenator which includes an array of semipermeable tubules which permit exchange of gases such as CO₂, CO, or O₂ but do not permit cells or proteins to cross. ECMO devices for humans that are currently in use contain within the oxygenator a large volume which facilitates the flow of blood through the array of tubules, which is referred to herein as the blood-and-gas tubule arrays (FIG. 1B). As in large animals and humans, the dense, high-volume environment of the oxygenators greatly limits penetration of light into the volume of the device.

As an initial step towards addressing this lack of light penetration, preliminary experiments were performed in which ECMO devices were re-constructed to produce a thin-layer version of an ECMO oxygenator-type device which allows external LED array sources to expose a sheet of flowing blood-and-gas tubules, similar to the types that are used as internal components of ECMO devices, to light. These experiments showed that light exposure can effectively remove CO by photodissociation in this arrangement and provided an indication that it would be feasible to use PECMO for removal of CO. While the prototype device was made large, flat, and thin in order to allow light penetration over a large area, we determined that what is needed to put PECMO into practice is a more practical and compact 3-D device, for example similar to devices used with standard ECMO systems, in which light enters deep into or is created within the volume of the device.

Accordingly, embodiments of a PECMO device are disclosed herein. The general approach is to modify an existing oxygenator design, which has already been optimized for gas exchange, to deliver light throughout the volume of the oxygenator in order to release CO from Hb and to promote diffusion of the released CO into the gas tubules and out of the system. While the description herein may refer to the PECMO device as an oxygenator or a modified oxygenator because the ECMO oxygenator was used as a starting point for the PECMO design, the primary purpose of the disclosed PECMO system is to remove CO from Hb, although the PECMO device can also provide oxygenation.

In various embodiments, light can be generated within a PECMO device (e.g., by placing a source within the interior of the PECMO device) or can be delivered from outside of a PECMO device (e.g., using a waveguide or other light-transmitting mechanism), or some combination of both. The options for generating light to deliver to blood within the device include but are not limited to LEDs and laser sources, which are preferred because of their efficiency and narrow waveband output. For the same reasons, these sources are preferred if the source is external to the PECMO device. In various embodiments, other possible light sources include xenon lamps, pulsed xenon flash lamps, and/or fluorescent lamps with appropriate wavelengths.

The interior of a PECMO device must be sterile, and therefore optical sources built into a device must also be sterile. Standard ECMO devices are intended for single use rather being sterilized and re-used. Placing light sources inside a PECMO therefore would mean that the light sources would also be disposable. While this is certainly possible, in various embodiments it is preferable to have the light source(s) external to the device. In addition, heat is generated even from the most efficient light sources. If light sources are placed within a PECMO device, the potential for thermal damage can place a limit on device design.

In various embodiments, delivery of externally-generated light into a PECMO device can be performed using a transparent medium that extends well into the interior of the PECMO, which is referred to herein as an “Optical Intrusion” (OI). While the examples below are directed to the use of OIs formed of solid materials, in various embodiments the OI transparent medium can be a gas or a liquid in addition to a solid. The transparent medium can be a waveguide such as a fiber optic, other solid-material transparent waveguides, or a transparent material extending into the interior of the PECMO device.

In one particular embodiment, a transparent sterile material can be used as the OI and one or more of such OIs can extend from the external wall(s) into the interior of the oxygenator device to deliver external light to the interior space of the housing. In various embodiments, the OI(s) can be made of a transparent plastic which may be similar, or identical, to that of the external wall, and in some embodiments the OI(s) may be integral to the external wall, for example made by a molding process (e.g., injection molding) that forms both the external wall and OI(s) as a single piece. In various embodiments, other materials that can be used to make the OIs include plexiglass/acrylic, polyethylene terephthalate, glass, polydimethylsiloxane (PDMS), and/or polycarbonate.

In other embodiments, the OI(s) can be made from optical fibers. In a hybrid version which effectively provides both externally- and internally-generated light, the OIs can include a fluorescent material (e.g., applied to a surface of the OI and/or integrated into the material that makes up the OI) that converts light from an external source which emits light within a given wavelength band to a different, longer-wavelength band within the PECMO device (see below).

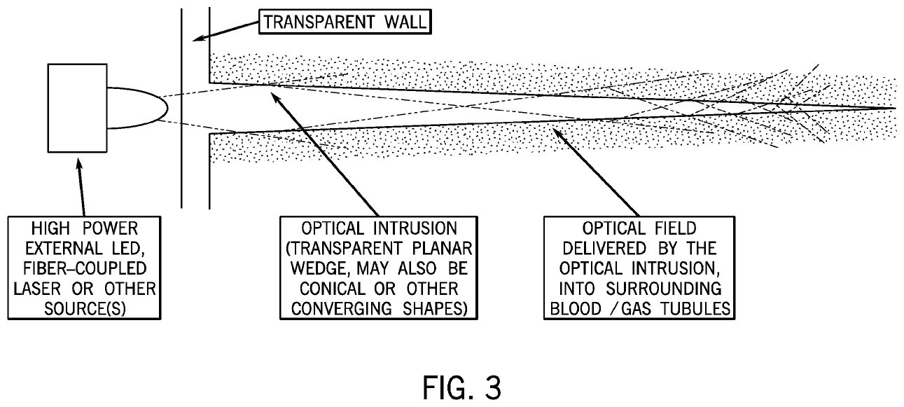

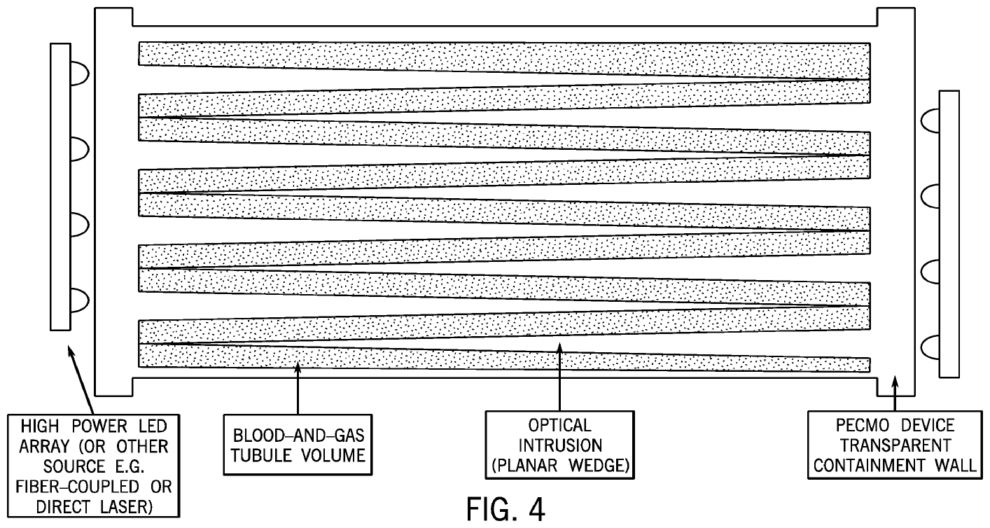

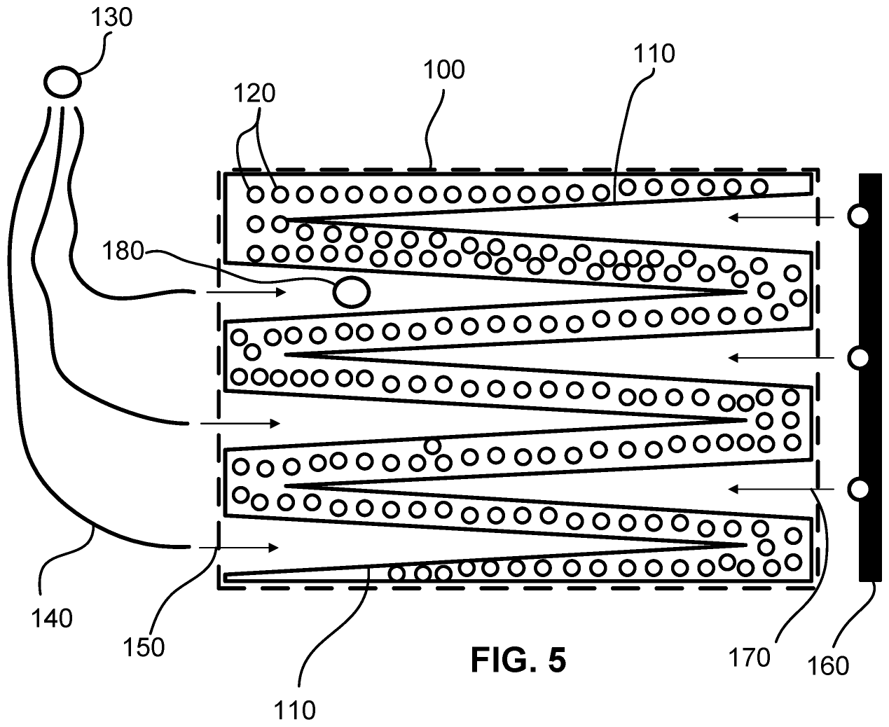

To achieve a compact PECMO device that allows light exposure throughout the active volume of the device, in some embodiments multiple OIs can be used to deliver light into multiple volumes of blood and gas exchange tubules. For simplicity and practicality of manufacture, in certain embodiments a preferred version uses transparent plastic OIs that are part of the external wall of the device (see FIGS. 3 and 4). Nevertheless, in other embodiments the OIs can be implemented as internally-extending cavities (e.g., hollow depressions into, or extensions of, the housing into the interior space, see FIG. 5), for example as planar wedges, conical shapes, or other (generally tapering) solid shapes that extend into the PECMO device. The examples shown in FIGS. 3 and 4, which are depicted in cross-section, can be conical, pyramidal, or planar wedge OIs that deliver external light from both surfaces of the wedge or all sides of the conical or pyramidal surface. Multiple planar wedge OIs can be arranged to provide a “sandwich” made up of alternating light-delivery and blood/tubule layers throughout the device as shown in FIGS. 4 and 5. Planar wedge OIs can deliver light from opposite sides of a PECMO device (FIGS. 4 and 5), achieving nominally uniform exposure deep within and throughout the device.

FIG. 3 shows a diagrammatic cross-section of a planar wedge OI showing some exemplary optical ray tracing (dashed lines) as well as the optical field (shaded region) arising from an external source. In this example, the OI is made of transparent plastic molded into the PECMO device external housing wall as a single unit. Coupling of light from within the OI occurs at its boundaries with the blood/gas tubule volume within the PECMO oxygenator device. Ray tracing is intended to show partial transmission and partial reflection as light encounters the boundary. Variations are also possible, for example the OI can be a convergent shape other than planar (e.g., conical or pyramidal) and its boundaries or faces may be either flat, convex, or concave. A combination of OI refractive index, OI shape, and source divergence largely determines the distribution of light delivery from the OI. One or more of these factors can be adjusted in the design. The blood/gas tubule compartment is within the device, external to the OI but in contact with the OI surfaces.

FIG. 4 shows an oxygenator housing having multiple planar-wedge OIs in cross-section, extending inward from the device external walls to deliver optical radiation into the volume of the device, which also contains blood/gas tubules surrounding the OIs. In this example, the small tubules are seen end-on, running in the gap between the OIs shown here in cross-section. The external light sources may be permanent equipment and need not be sterile. For example, the sources may be part of a re-useable container or housing for a disposable PECMO device. The housing may also contain pumps, temperature controllers, heat exchangers and/or other equipment components.

FIG. 5 provides a diagram showing a cross-section of a housing 100 (shown with dashed lines) of a PECMO device in which the OIs 110 are hollow cavities extending into the interior of the housing 100, where the OIs can be extended wedges, cones, or pyramids (e.g., having 3, 4, 5, or other number of sides). As shown in FIG. 5, in some embodiments multiple OIs 110 can be inserted into the interior of the housing 100 and the OIs 110 can extend from one or more sides of the housing 100. In certain embodiments, the OIs 110 can be arranged such that there is a characteristic spacing between the opposing faces of adjacent OIs which optimizes light penetration and subsequent release of CO from Hb, which in one particular embodiment may be 4 mm when the light has a wavelength of approximately 630 nm (see below).

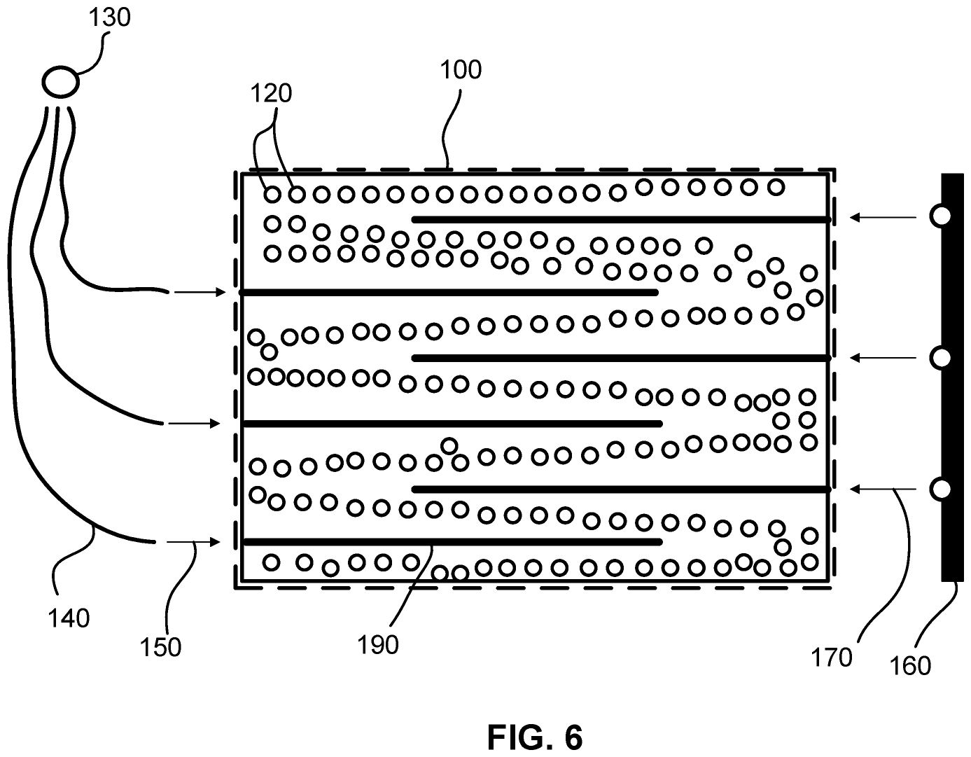

The OIs 110 are surrounded by an array of gas-permeable tubules 120 (shown in cross-section as a series of circles) which are immersed in blood from a patient. Gases such as O₂ diffuse from the tubules 120 and exchange with gases such as CO and CO₂ released from the Hb in blood (red blood cells) within the interior of the housing. Light from a light source 130 (e.g., a laser or LED) is transmitted by a waveguide 140 (e.g., an optical fiber) and directed 150 into the cavities of the OIs 110, where the light crosses the surface of the OIs (e.g., by refraction) into the blood and interacts with compounds such as HbCO, possibly causing dissociation of the CO from the Hb such that the CO can diffuse to one of the tubules 120 and exit the system. Light can also be emitted from an array of sources 160 (e.g. lasers or LEDs) where it can be directed 170 into the cavities of the OIs 110. Further, a light source 180 can be located within one or more OIs to emit light into the adjacent interior of the housing. In some embodiments a series of fiber optic OIs 190 (FIG. 6) can extend into the interior of the housing 100 instead of or in addition to the wedge-shaped OIs (e.g., as shown in FIG. 5). The fiber optic OIs 190 can extend perpendicular to the gas-permeable tubules 120 (as shown in FIG. 6) and/or parallel to the tubules 120. In various embodiments the fiber optic OIs can be covered with a clear material to avoid direct contact of the optical fibers with blood.

The shape and placement of OIs are important for effective performance of the PECMO device because overall efficiency of the device is highest when light is delivered and absorbed by HbCO at or near to the blood-gas exchange interface of each tubule. Photo-dissociated CO molecules can rapidly re-bind to empty binding sites on Hb rather than diffusing to the gas exchange interface and being subsequently removed from the device by gas flow. Both HbO₂ and HbCO spontaneously dissociate and then re-associate in a dynamic equilibrium. However, as mentioned above HbO₂ spontaneously dissociates far more easily than HbCO, which strongly favors the accumulation of HbCO when light is not present. By preferentially photodissociating HbCO more than HbO₂, light changes the dynamic equilibrium to be more in favor of HbO₂. The local rate of HbCO photodissociation per unit volume of blood is proportional to the rate of light absorption per unit volume by HbCO, which is directly proportional to local irradiance. In preliminary experiments using the prototype, the rate of CO removal was observed to be proportional to the incident optical irradiance—indicating that irradiance was a limiting factor in the prototype design rather than other factors such as oxygen or blood flow rates. This indicates that better delivery of higher optical power will improve performance well over that of the prototypes.

As noted previously, the CO locally released by photodissociation of HbCO within a PECMO can re-bind to available sites on Hb. Thus, a photon absorbed by HbCO is in effect “wasted” if the released CO molecule does not reach a gas exchange tubule before the CO molecule re-binds to an empty Hb site. When photodissociation of HbCO occurs in blood that is too far away from a gas exchange site (i.e., in a blood volume far away from the tubules of a PECMO device), then the dissolved CO is likely to bind to empty Hb sites before it reaches the gas exchange site. In theory it may be possible to estimate how far away from a tubule photodissociation should occur, for example based on the diffusion rate of dissolved CO and the rate at which Hb binding sites become available to it. There are many factors that affect the dynamic equilibria and diffusion gradients involved for Hb, HbCO, HbO₂, O₂ and CO, including but not limited to photodissociation rate and sites, gas diffusion, temperature, the complex structure and kinetics of cooperative hemoglobin binding, conformational changes in hemoglobin, PECMO tubule structure, flow rates for blood and gases in the device, and more. Despite decades of research a complete model does not exist for competitive O₂ and CO binding kinetics and, given the novelty of this concept, no model yet exists for binding kinetics within a PECMO device.

However, a simplified way to estimate how far away photodissociation should occur from the gas exchange site in an efficient PECMO device is that the distance should ideally be about equal to or less than that for a molecule of CO to diffuse in blood during the time for which the CO molecule is likely to encounter an open Hb binding site. In a PECMO device the gas exchange interface is always at the capillary tubules. In the absence of a suitable model, we can use the kinetics of gas exchange in lung as a guide. The alveoli in the lungs are gas-filled sacs about 0.2 mm in diameter, similar in size to the tubule diameter of a PECMO device. Complete gas exchange occurs in lung alveoli in ˜0.5 seconds. During this time, dissolved CO will diffuse a distance of about 0.4 mm (the diffusion constant is about 2.4×10⁻⁵ cm²/s). In other words, we expect that photons that are absorbed within about 0.4 mm of the closest tubule have a good chance of liberating a CO molecule which actually leaves the blood and is removed from the device. In practice this amounts to making devices in which the spacing between tubules in the active part of the device is about 0.8 mm or less. This is not an absolute requirement but nevertheless is desirable for efficiency. The tubules of current ECMO devices are tightly packed and meet this condition.

Wavelength of light is an important design consideration. The quantum yield for HbCO photodissociation is independent of wavelength, so there is no photochemical reason to choose one wavelength over another. Furthermore, the visible light absorption spectra for HbO₂ and HbCO are nearly identical, and HbCO exhibits far lower absorption of near-IR light, such that there is no “best” wavelength to optimize HbCO absorption over HbO₂ absorption. Therefore, in various embodiments the wavelength of light was chosen to optimize the depth of penetration into the mixture of blood and tubules within a PECMO device as well as based on the availability of efficient, high average power practical light sources that provide output at a particular wavelength. Fortunately, there are practical, efficient light sources available which emit light across the visible spectrum, including sources that emit blue, green, and/or orange/red light.

Penetration depth in blood is determined by optical absorption and scattering; to a first approximation throughout most of the visible spectrum, the penetration depth is d˜μₐ⁻¹ where μₐ is the optical absorption coefficient of the blood-and-tubules media. The value of μₐ is given by the absorption spectra and concentration of the various species of hemoglobins present in blood. Specifically in a PECMO device μₐ˜2.3 ecF, where e is the wavelength-dependent molar extinction coefficient of the combination of hemoglobins in blood, c is the concentration of hemoglobins in blood, and F is the volume fraction of blood in the light-exposed interior of the device. The factor F accounts for the presence of gases within the device. For example, if gas-filled tubules occupy 60% of the device volume, the value of F is 0.4. The value of c in healthy adults is about 2×10⁻³ moles/liter. In a practical device, one or more wavelengths are chosen which penetrate about half of the distance between OIs in the interior of the PECMO, such that all of the blood/tubule volume between OIs receives approximately uniform light exposure. In our preliminary studies we chose an LED waveband at about 630 nm (˜10 nm FWHM bandwidth), which corresponds in whole venous blood to e˜2000 cm⁻¹ M. If F˜0.5, the value of μₐ is about 5 cm⁻¹ and light therefore penetrates about 2 mm through the blood/tubule volume inside a PECMO device. Using the simplest design of OIs made up of planar wedges, if the source emits light at around 630 nm the wedge faces of the OIs could be spaced about 4 mm apart and still provide nominally uniform exposure to the blood/tubule volume between them. In various embodiments, the light source emits visible (e.g. 400-700 nm) or infrared (e.g. 700 nm-1 mm) light. In other embodiments, the light source emits light of at least 400 nm, at least 500 nm, or at least 600 nm. In some embodiments, the light source emits light between 600-700 nm, between 620-640 nm, or between 625-635 nm.

As noted above, the optical intrusions (OIs) can be various shapes including wedges, cones, or pyramids (having 3, 4, 5, 6 or other numbers of sides). Delivery of a nominally uniform optical field by the OI is determined by a combination of device shape, device refractive index, and divergence of the external light sources. Ray-tracing programs are available which can be used to optimize the OIs for a given source and PECMO device size. Optical scattering by the multiple gas-blood interfaces between OI is more of an advantage than a disadvantage, because multiple scattering tends to produce a uniform local optical distribution. Monte Carlo models exist which describe the optical distribution within complex turbid media and allow the source and device geometry to be varied, which aids in detailed device design. For example, see SL Jacques, Photochem. Photobiol. 67:23-32, 1998, incorporated by reference herein in its entirety.

In some embodiments, there may be a fluorescent dye incorporated into the OI, and the external light source may be at a wavelength exciting fluorescence emission at a longer desired emission wavelength band, for example emission of yellow, orange and/or red visible light wavelengths from an excitation source that is blue or green. There are many high-efficiency fluorescent acrylic plastic materials available that can be formed into an OI. For example Rhom and Haas Plexiglas R123 emits a waveband near 630 nm when excited by blue or green light. Alternatively, the OI may be fiber optic waveguides in which the cladding material is fluorescent, such that evanescent wave coupling from the core to cladding excites fluorescent emission from the cladding layer.

In another embodiment, optical fibers can be used as the OIs, as shown in FIG. 6. For example, side-emitting optical fibers can be placed within the volume of the PECMO. The fibers can be aligned parallel, perpendicular, or at some other angle to the gas-exchange tubules. In a preferred version, the fibers are spread out within the device volume, mixed in parallel with the gas-exchange tubules. The optical fibers are gathered together and/or spliced to form an external port for light delivery. The fibers may be made from optically transparent plastic or glass. In a preferred version, cladding of the fibers is made of a fluorescent material that converts a shorter wavelength(s) of optical power within the fiber core, to emission at a longer desired wavelength(s) region. For example, blue laser light can be converted to longer-wavelength green, yellow, orange, or red light. An advantage of optical fibers is that they can very efficiently transport large optical power through a very small core, providing high optical power within a PECMO device without adding much volume to the device.

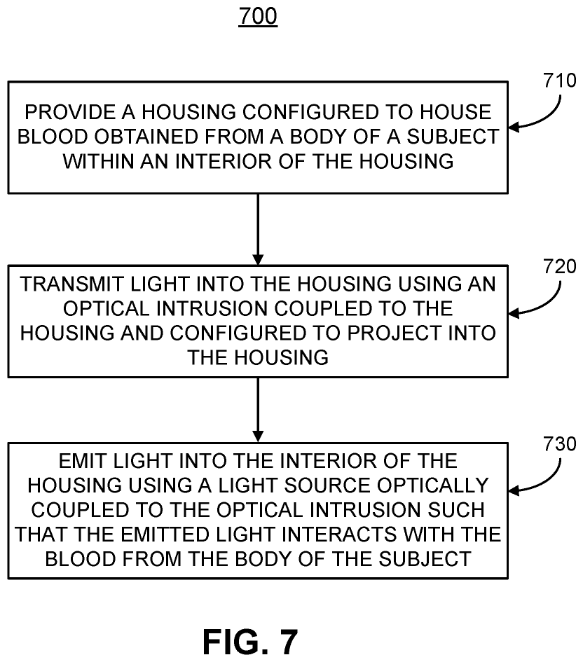

FIG. 7 shows an example 700 of a process for removing CO from blood in accordance with some embodiments of the disclosed subject matter. As shown in FIG. 7, at 710, process 700 can provide a housing configured to house blood obtained from a body of a subject within an interior of the housing. The housing can include a plurality of gas-permeable tubules disposed within the interior of the housing. At 720, process 700 can transmit light into the housing using an optical intrusion coupled to the housing and configured to project into the housing. Finally, at 730, process 700 can emit light into the interior of the housing using a light source optically coupled to the optical intrusion such that the emitted light interacts with the blood from the body of the subject.

It should be understood that the above described steps of the process of FIG. 7 can be executed or performed in any order or sequence not limited to the order and sequence shown and described in the figures. Also, some of the above steps of the processes of FIG. 7 can be executed or performed substantially simultaneously where appropriate or in parallel to reduce latency and processing times.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto

What is claimed is:

1. An apparatus for removing CO from blood, the apparatus comprising:
a housing configured to house blood obtained from a body of a subject within an interior of the housing;
a plurality of gas-permeable tubules disposed within the interior of the housing;
an optical intrusion coupled to the housing and configured to project into the housing,
the optical intrusion configured to transmit light into the interior of the housing; and
a light source optically coupled to the optical intrusion, the light source being configured to emit light which is coupled via the optical intrusion into the interior of the housing such that the emitted light interacts with the blood from the body of the subject.

2. The apparatus of claim 1, wherein the optical intrusion comprises a cavity within the interior of the housing, and
wherein the light source is disposed within the cavity.

3. The apparatus of claim 1, wherein the optical intrusion is configured to transmit light from outside the housing into the interior of the housing.

4. The apparatus of claim 3, wherein the light source is outside the housing and adjacent to the optical intrusion.

5. The apparatus of claim 3, wherein the light source is outside the housing and coupled to the optical intrusion by a waveguide.

6. The apparatus of claim 1, wherein the optical intrusion comprises a fiber optic extending into the interior of the housing.

7. The apparatus of claim 1, wherein the optical intrusion comprises a tapered structure extending into the interior of the housing.

8. The apparatus of claim 7, wherein the tapered structure is hollow.

9. The apparatus of claim 7, wherein the tapered structure is solid.

10. The apparatus of claim 7, wherein the tapered structure is part of the housing.

11. The apparatus of claim 7, wherein the tapered structure has a shape of at least one of a wedge, a cone, or a pyramid.

12. The apparatus of claim 1, wherein the optical intrusion comprises a plurality of tapered structures.

13. The apparatus of claim 12, wherein each of the plurality of tapered structures is spaced 4 mm apart.

14. The apparatus of claim 1, wherein the optical intrusion comprises a fluorescent material.

15. The apparatus of claim 1, wherein the light source comprises at least one of an LED or a laser.

16. The apparatus of claim 1, wherein the light source emits light between 400-700 nm.

17. A method for removing CO from blood, the method comprising:
providing a housing configured to house blood obtained from a body of a subject within an interior of the housing,
a plurality of gas-permeable tubules disposed within the interior of the housing;
transmitting light into the housing using an optical intrusion coupled to the housing and configured to project into the housing; and
emitting light into the interior of the housing using a light source optically coupled to the optical intrusion such that the emitted light interacts with the blood from the body of the subject.

18. The method of claim 17, wherein the optical intrusion comprises a cavity within the interior of the housing, and
wherein transmitting light into the housing further comprises:
transmitting light into the housing using the light source disposed within the cavity.

19. The method of claim 17, wherein transmitting light into the housing further comprises:
transmitting light from outside the housing into the housing.

20. The method of claim 19, wherein the light source is outside the housing and adjacent to the optical intrusion, and
wherein transmitting light into the housing further comprises:
transmitting light into the housing using the light source outside the housing and adjacent to the optical intrusion.

21. The apparatus of claim 19, wherein the light source is outside the housing and coupled to the optical intrusion by a waveguide, and
wherein transmitting light into the housing further comprises:
transmitting light into the housing using the light source outside the housing and coupled to the optical intrusion by a waveguide.

22. The method of claim 17, wherein the optical intrusion comprises a fiber optic extending into the housing, and
wherein transmitting light into the housing further comprises:
transmitting light into the housing using the fiber optic extending into the housing.

23. The method of claim 17, wherein the optical intrusion comprises a tapered structure extending into the housing, and
wherein transmitting light into the housing further comprises:
transmitting light into the housing using the tapered structure extending into the interior of the housing.

24. The method of claim 23, wherein the tapered structure is hollow.

25. The method of claim 23, wherein the tapered structure is solid.

26. The method of claim 23, wherein the tapered structure is part of the housing.

27. The method of claim 23, wherein the tapered structure has a shape of at least one of a wedge, a cone, or a pyramid.

28. The method of claim 17, wherein the optical intrusion comprises a plurality of tapered structures, and
wherein transmitting light into the housing further comprises:
transmitting light into the housing using the plurality of tapered structures.

29. The method of claim 28, wherein each of the plurality of tapered structures is spaced 4 mm apart.

30. The method of claim 17, wherein the optical intrusion comprises a fluorescent material, and
wherein emitting light into the interior of the housing further comprises:
emitting light into the interior of the housing using the fluorescent material.

31. The method of claim 17, wherein the light source comprises at least one of an LED or a laser, and
wherein transmitting light into the housing further comprises:
transmitting light into the housing using at least one of the LED or the laser.

32. The method of claim 17, wherein the light source emits light between 400-700 nm, and
wherein transmitting light into the housing further comprises:
transmitting light into the housing using the light between 400-700 nm.
